(19) United States
(12) Patent Application Publication
(10) Pub. No.: US 2014/0213976 A1
(43) Pub. Date: Jul. 31, 2014

(54) Drug Delivery Infusion Set with Manual Pump

(71) Applicant: INSULINE MEDICAL LTD, Petach Tikva (IL)

(72) Inventors: Gabriel Bitton, Jerusalem (IL); Ron Nagar, Tel Aviv-Yafo (IL)

(21) Appl. No.: 14/122,440

(22) PCT Filed: May 30, 2012
(86) PCT No.: PCT/IL2012/000211

§ 371 Date: Apr. 1, 2014

Related U.S. Application Data
(60) Provisional application No. 61/491,234, filed on May 30, 2011.

(51) Int. Cl. A61M 5/142; A61M 5/00

(52) U.S. Cl. CPC A61M 5/14244; A61M 5/003
USPC 604/151

(57) ABSTRACT

The subject matter described herein relates generally to systems and methods for delivering one or more drugs to a user while applying treatment to the infused drug site. In particular, the subject matter described herein relates to device, systems and methods for drug delivery using manual pump with an infusion set that includes one or more treatment sources to improve effectiveness of the injected medicament

### FIELD OF THE INVENTION

The subject matter described herein relates generally to systems and methods for delivering one or more drugs to a user while applying treatment to the infused drug site. In particular, the subject matter described herein relates to device, systems and methods for drug delivery using manual pump with an infusion set that includes one or more treatment sources to improve effectiveness of the injected medicament.

### BACKGROUND OF THE INVENTION

Infusion pumps and other devices are used regularly for subcutaneous injections of therapeutic fluids, drugs, proteins, and other compounds for humans or animals. Such delivery systems and methods are used also for insulin delivery. A conventional infusion pump is an infusion device specifically adapted for infusing a drug to the subcutaneous tissue using an infusion set connecting the drug reservoir within the pump to a catheter adhered to the skin. The pump and infusion set can be disconnected and reconnected to the catheter leaving only the catheter on the skin when the user desires, for example, before swimming or taking a shower or other activities that might harm the pump or when the pump might disturb the activity. Typically, the infusion set including the catheter is replaced regularly, usually every three days.

Infusion pumps may deliver medicament around the clock. For example, an infusion pump may provide a slow drip of medicament that may be increased by the user or by other input before an event that requires a large amount of medicament. For diabetics, insulin infusion pumps deliver basal insulin around the clock, in addition to a bolus dose of insulin, which may be adjusted by the user, to be delivered by the infusion pump before, during, or immediately after a meal. It is desired that the bolus dose of drug or insulin reaches the circulatory system quickly, and provides an amount of insulin that matches the amount of carbohydrates consumed by the patient. This fast acting delivery to the circulatory system is not required for the basal drip.

It is estimated that a large number of infusion pumps are being used today by hundreds of thousands of diabetics worldwide. Such pumps are generally automatic pumps that are expensive and therefore many diabetics do not have access to such technologically heavy and expensive drug delivery pumps.

Many medical treatment systems and methods involve drug delivery systems that employ subcutaneous infusions of therapeutic fluids, drugs, proteins, and other compounds. Such delivery systems and methods, especially in the area of insulin delivery, have made use of subcutaneous catheters and continuous subcutaneous insulin infusion (CSII) pumps. In conventional insulin pumps, the pump is configured to be attached to a disposable thin plastic tube or a catheter through which insulin passes into the tissue. The catheter can be inserted transcutaneously, typically through the skin of the patient's abdomen, and is changed every two to three days. New types of insulin pumps, such as the OmniPod pump manufactured by Insulet Corporation, do not have an external catheter and, instead, a catheter port is embedded into the pump mechanism.

In many instances, patients require insulin delivery around the clock to keep proper levels of glucose in their blood. Insulin can be delivered at a basal rate or in bolus doses. The basal rate represents insulin that is continuously delivered to the patient. Such a continuous delivery of insulin intends to maintain the blood glucose level within a desired range between meals and overnight. A bolus dose is an amount of insulin delivered to the patient according to food intake at meals, particularly carbohydrates. When patient consumes food, his or her levels of glucose rise.

Typically pumps are expensive, have several parts connected by a tube, include electronics and rely on battery power to operate. Even newer patch pumps are expensive and are expensive and rely on battery power to operate. On the other there are other methods to deliver insulin which are simpler for example insulin pens and insulin syringes which deliver a single dose of insulin, however such delivery methods require continuous patient intervention and/or involvement.

When a diabetic patient consumes food, his or her level of glucose rises. Unfortunately, existing subcutaneous injection devices, including infusion pumps, are incapable of quickly matching or preventing the rise of blood glucose. The delay in such matching is also true in case of “rapid-acting” insulins. Some of the reasons for this delay include a lag in the absorption of insulin from the time insulin is injected at the injection site to the time it takes for complex insulin molecules to break down into monomers.

Additionally, since blood glucose levels rise shortly following the meal, the delay in matching insulin to the rising glucose level may cause a post prandial hyperglycemic event (i.e., when the blood glucose level is above normal) to occur. Furthermore, after a certain period of time passes after a meal (e.g., 2-3 hours), the blood glucose level could drop while the insulin concentration in the blood rises followed by the peak of the systemic insulin effect. This may cause a hypoglycemic event (i.e., when the blood _{g}lucose level is below normal) to occur. Both hyperglycemic and hypoglycemic events are highly undesirable. Additionally, since local blood perfusion at the insulin injection region, including that of insulin infusion set connected to insulin infusion pump, has large variability, depending on the ambient temperature and other parameters, it induces large variations to the delay of the peak of time profile of the insulin action. Those variations in the insulin peak action period further increase the variability in the blood glucose level.

Additionally, it is known that certain drugs, including insulin, are growth hormones. These drugs, when injected several times at the same location, can cause local cell growth, causing Lipohypertrophy. Using regular infusion sets and infusion pumps, the drug may be infused several times per day, and possibly for several days, at the same location, which could result in Lipohypertrophy. Increasing local blood perfusion at the injection site to promote drug uptake to the circulatory system may reduce the unwanted Lipohypertrophy phenomenon.

Therefore, it is desirable to provide a system and method that provide efficient and rapid uptake of infused drug to the circulatory system when the drug is infused by an infusion pump.

Additionally, it is desirable to provide a system, device and method that provide treatment to maintain a normal blood glucose level and prevent or reduce hyperglycemic and hypoglycemic events, and reduce Lipohypertrophy.

### SUMMARY OF THE INVENTION

There is an unmet need for, and it would be highly useful to have, a drug delivery infusion set and drug delivery pump that provides a low cost mechanical alternative to state of the art infusion set pumps while providing improved drug delivery profile, and in particular optimizing insulin drug delivery to diabetics.

It would be desirable to have a modified pump which has the benefits of the pump such as bolus and basal operation modes while still keeping the simplicity of insulin injection. It would also be desirable to have in this modified pump elements for tissue treatment that will enable faster and more repeatable mode of insulin delivery into the body.

The present disclosure relates to systems, devices, and methods for infusing one or more drugs, substances, and/or chemicals to a user that improve the effectiveness of drug delivery upon infusion of the drug(s). The systems, devices, and methods may provide an additional treatment to a tissue region where the drug is delivered. In some embodiments, the treatment is utilized to improve the drug delivery process by improving the drug's pharmacokinetic and/or pharmacodynamic profile. The treatment may include, but is not limited to, methods and devices described in PCT/IB2008/051049, PCT/IB2008/051044, and PCT/IB2009/007600, which are incorporated herein by reference. In some embodiments, the treatment may come in various forms, for example, including an analgesic, vasodilator or the like. In some embodiments, the treatment may be any form of treatment that leads to improved vasodilatation of the tissue being injected. To improve the drug's pharmacokinetic and/or pharmacodynamic profile, the treatment may optionally include, but not limited to, exposing the tissue region to energy, radiation, heat, mechanical vibrations, suction, massaging, acoustic stimulation, electrical stimulation, injection of one or more additional substances, or any combination of the above. Each treatment type may optionally have a separate protocol in order to evoke the necessary reaction such as vasodilatation or the like.

Within the context of this application the term “treatment” and/or “treatment element” is to refer to any treatment type or a combination of treatment types that may be applied to an injection area to most preferably optimize drug delivery profile by directly or indirectly improving the delivery and absorption of the drug, for example insulin, to improve the drug's pharmacokinetic and for pharmacodynamic profile, optionally and preferably by improving vasodilatation of the tissue about the injection site. A treatment element and/or treatment or combination of treatment elements may come in various forms, for example including but not limited to an analgesic, vasodilator, or the like. Optionally, the treatment may be any form of treatment that leads to an improved vasodilatation of the tissue in and about the injection site, where the treatment, may for example include but is not limited to, exposing the tissue region to an energy, radiation, heat, mechanical vibrations, suction, massaging, acoustic stimulation, electrical stimulation, injection of an additional substance(s), or any combination of the above to improve drug's pharmacokinetic (‘PK’) and for pharmacodynamic (‘PD’) profile. Optionally an applied treatment may induce vasodilatation through neural stimulation of the tissue around the drug injection site. The neural stimulation can be induced by thermal stimulation. The human neural response to thermal stimulation includes several mechanisms such as the Nociceptive Axon Reflex that induce vasodilatation among other effects.

Optionally, an induced neural response, such as the nociceptive axon reflex, also optionally induces widening of the capillary pores and increasing the capillary wall permeability. This effect is also significant for improving the absorption of the drug through the capillary wall.

Optionally and preferably a treatment and/or a treatment element may be provided and/or applied to the injection site before, during or after administration and/or injection of the drug.

Optionally and most preferably the treatment element is controllable and may be provided in the form of a heater. Most preferably a drug's temperature sensitivity can be accounted for so as to avoid protein denaturisation.

Optionally, in some embodiments, the applied treatment induces vasodilatation through neural stimulation of the tissue around the drug infusion site through the infusion set and infusion pump. The neural stimulation can e induced by thermal stimulation. The human neural response to thermal stimulation includes several mechanism such as the Nociceptive Axon Reflex that induces vasodilatation, among other effects.

Optionally, in some embodiments, the induced neural response, such as the nociceptive axon reflex, also optionally induces widening of the capillary pores and increasing the capillary wall permeability. This effect is also significant for improving the absorption of the drug through the capillary wall.

Optionally, in some embodiments, the applied treatment may lead to a reduction in the variability of the drug absorption in the blood or lymph system, and its local and systemic effects. For example, heating the tissue region in the vicinity of the area of drug delivery through an infusion set catheter connector to an infusion pump, to a preset regulated temperature during and/or after the drug infusion and absorption into the blood may cause local blood perfusion at that region to become more reproducible and the drug absorption process more uniform and reproducible as well. Also, by reducing the delay between drug infusion into the tissue through the infusion set catheter connected to infusion pump, and absorption into the blood system, the variability of drug action induced by the delayed profile can be reduced. In some embodiments, the temperature of the region adjacent to the infusion region through the infusion set catheter connected to infusion pump is regulated for longer periods, but this may require additional energy source capacity and increase weight. Therefore, to minimize the energy source size, the heating period or heating temporal profile should be optimized in relation to the period of the drug infusion and absorption into the blood. In some embodiments in which the treatment utilized is, for example, heat, the drug interaction with the treatment substance or type will be preferably taken into consideration and avoided. In some embodiments, a drug's temperature sensitivity will be accounted for so as to avoid, for example, protein denaturisation. For example, since insulin is a temperature sensitive protein, a treatment protocol, which includes heat, may be limited so as to ensure the efficacy of the delivered drug when it contains insulin. To achieve this, the treatment protocol may control the temperature or the location of the treatment delivery site so as to not damage the drug. For instance, heating some types of insulin above 37° C. may damage the insulin. Accordingly, in some embodiments, the tissue around the infusion site is heated to induce the desired neural response without heating the insulin itself above 37° C. For example, heating the tissue at a distance of 10 mm around the injection site to 38.5° C. provides a significant vasodilatation without heating the injected insulin above 37° C. It is also possible to heat with a spatial distribution of temperature around the center of the infusion set catheter connected to an infusion pump in a manner such that a region away from the center is heated at a higher temperature while a region closer to the center is heated at a lower temperature.

In some embodiments, the device further includes a treatment element for performing a treatment on the mammal. The device may also include a controller for controlling the treatment element. For example, the controller may be configured to active the treatment element based on information regarding the therapeutic substance, which may be stored, for example, with the reservoir containing the therapeutic substance. The device may further include means for determining if the infusion set has been previously used and/or a number of times that the treatment element has been activated. For example, the means may include one or more fuses which is burned each time the treatment element is activated. Within the context of this application the term “single use” is interchangeable with the terms “single use time frame” or “single use period” or “single use period protection” refer to mechanical, electronic, or the like means utilized to ensure that a disposable unit and/or portion intended for single use is rendered non-operational and/or non-functional beyond the single user period intended for the device, unit, member or portion. Optionally and preferably a single use period protection utilized is defined according to the single use item. For example on disposable injection site unit according to the present invention may have a single use period of 24 hours while the single use period of disposable injection port according to an optional embodiment of the present invention may be defined to have a single use period of up to 3 days. Optionally the single use period protection means include mechanical and/or electronic means may be disposed on the single use and/or disposable member itself or by way of interaction (mechanical or electronic) with other non-disposable members. Optionally single use period protection means may for example include but is not limited to mechanical integrity of housings, connectors, adhesive, electrical contacts, electrical communication, time based interactions, or the like.

An optional embodiment of the present invention provides a device for delivering a therapeutic substance to the body of a mammal, comprising:

a treatment element for modifying the pharmacodynamic or pharmacokinetic properties of the therapeutic substance;

a reservoir for storing the therapeutic substance,

a manual infusion pump functionally associated with the reservoir for dispensing the contents of the reservoir; wherein the reservoir may be in fluid communication and coupled with an infusion set catheter with a coupling member; the infusion set comprising:
a catheter disposed in a catheter housing, the housing having an adhesive layer for coupling to a body, and wherein the catheter may be in fluid communication and coupled with the reservoir about the coupling member.

Optionally the coupling member comprises a first member disposed about the catheter and a second member disposed about the reservoir.

Optionally, the device may be rendered non-functional unless the first and second coupling member are properly coupled forming a fluid communication channel between the reservoir and the catheter.

Optionally, the first and second coupling members are self-sealing.

Optionally, the treatment element may be disposed about the infusion set catheter housing or the a manual infusion pump.

Optionally the treatment element may be provided in the form of a heating element. Optionally, the device may further comprise an electronic module for controlling the treatment element. Optionally, the controller may be configured to activate the treatment element based on information regarding the therapeutic substance.

Optionally the electronic module comprises one or more fuses which may be burned each time the treatment element may be activated.

Optionally, the controller executes a treatment protocol based on the information regarding the therapeutic substance.

Optionally, the controller may be configured to automatically activate the treatment profile element based on information of therapeutic substance volume delivered.

Optionally the reservoir may be provided in the form of a single use cartridge.

Optionally, the manual infusion pump provides for activating the treatment element.

Optionally, the manual infusion pump provides for determining the amount of drug dispensed from the reservoir.

Optionally the therapeutic substance may be insulin.

Optionally the infusion set comprises identifying means that uniquely identifies the infusion set.

Optionally the device and system and further provided with a drug safety element provided to avoid accidental infusion. Optionally the safety element comprises an Optionally, the coupling member further comprises and/or provides for a drug safety element to safeguarding against accidental drug infusion.

An optional embodiment of the present invention provides a system for delivering a therapeutic substance to the body of a mammal, comprising an auxiliary device in communication with the infusion set and manual pump according to optional embodiments of the present invention. Optionally, the auxiliary device may be selected from the group of mobile communication device, blood glucose monitor or computer. Optionally, the auxiliary device determines the amount dispensed from the reservoir.

According to some embodiments, the infusion pump may be provided of at two units and/or parts, comprising a disposable part and a reusable part. The disposable part including a catheter provided to deliver a drug into the body. The catheter comprising of a cannula which penetrate into the body tissue, and a biocompatible adhesive that mechanically secures the catheter to the body of a user. The reusable unit comprises a mechanical or electrical means to infuse a certain amount of insulin, most preferably in the form of a mechanical and/or manual pump, into the body upon demand or at a constant slow rate. The reusable part may further include a replaceable drug cartridge.

In some embodiments, optionally, the disposable unit may further include a treatment element to treat the tissue, most preferably to increase local blood perfusion so improve drug delivery into the tissue. Optionally, the system may include an insertion device to enable insertion of the catheter into the body as known in the art.

In some embodiments the infusion pump may include a manual controller and/or switch and/or knob where each turn will result in a delivery of one part of the drug. This part can be as small as 0.1 IU. Optionally turning the manual switch wind a member, for example a string, which after approving the drug delivery will infuse the drug into the tissue. Optionally use of the manual switch may produce a audible cue or sound for example a click, for each step which is a sign of the amount of the drug selected for infusion.

Optionally, in some embodiment the treatment element may be part of the disposable unit and may comprise include a heating element, a control unit and a power source. Optionally, the disposable portion may be displaced and/or removed when a need for drug infusion or tissue treatment is needed. Optionally and preferably the design of the heating element is such that when connecting the reusable part the heating element comes in good contact with the tissue.

Optionally, in some embodiment the drug cartridge may be part of the disposable unit and the reusable unit may be used to drive the drug out of the cartridge into the tissue by applying external pressure to the cartridge. For example, the cartridge may be a circular cartridge with a cap top which may be advanced when pressure is applied to it, similar to the mechanism in injection pens.

Optionally and preferably, the device and system may also include a securing element to avoid accidental infusion, optionally by way of mechanical and/or electrical means. For example an approval button to approve the delivery of the selected drug to be infused.

Optionally, in some embodiments the device may comprise communication capabilities and he can communicate with external display and communication unit and it may be able to display on that unit the amount of insulin to be infused. The external unit may also be used to approve the infusion of the selected amount of drug.

In some embodiments the knob and/or manual controller may only select the amount of drug to be infused by allowing a stopper to go further away and the actual physical infusion is done by applying pressure at the drug infusion site on the catheter in a similar manner that injection pens are used.

Details of one or more variations of the subject matter described herein are set forth in the accompanying drawings and the description below. Other features and advantages of the subject matter described herein will be apparent from the description and drawings, and from the claims.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or steps manually, automatically, or a combination thereof.

Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

Optionally any device featuring a data processor and/or the ability to execute one or more instructions may be described as a computer, including but not limited to a PC (personal computer), a server, a minicomputer, a cellular telephone, a smart phone, a PDA (personal data assistant), a pager. Any two or more of such devices in communication with each other, and/or any computer in communication with any other computer, may optionally comprise a “computer network”.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIG. 1A is a schematic block diagram of an exemplary drug delivery infusion set with low cost pump according to some embodiments of the present disclosure.

FIG. 1B is a schematic block diagram of an exemplary drug delivery infusion set with low cost pump according to some embodiments of the present disclosure.

FIG. 1C is a schematic block diagram of an exemplary drug delivery infusion set with low cost pump according to some embodiments of the present disclosure.

FIG. 1D is a schematic block diagram of an exemplary drug delivery infusion set with low cost pump according to some embodiments of the present disclosure.

FIG. 1E is a schematic block diagram of an exemplary drug delivery infusion set with low cost pump according to some embodiments of the present disclosure.

FIG. 2A-E show an illustrative schematic diagram of an exemplary drug delivery infusion set with low cost pump according to some embodiments of the present disclosure.

### DETAILED DESCRIPTION OF EMBODIMENTS

The principles and operation of the present invention may be better understood with reference to the drawings and the accompanying description. The following figure reference labels are used throughout the description to refer to similarly functioning components are used throughout the specification hereinbelow.
5 drug delivery infusion set with low cost pump;
6 treatment element;
8 drug or medicament reservoir;
10 Reusable Unit;
12 Drug delivery pump;
15 coupler;
20 Disposable Unit;
22 Infusion cannula;
24 Adhesive surface;
30 Electronic Module;
32 Controller;
34 Communication module;
36 Power supply module;
50 Auxiliary device;
100 infusion set;
110 cannula;
110i entry port;
120 adhesive layer and laminate;
130 catheter housing;
140 rotating circular disc;
150 functional unit member;
160 drug reservoir;
170 drug reservoir cavity;
180 connection exit port;
190 plunger;
200 manual pump member;
210 treatment element;
220 electronics module;

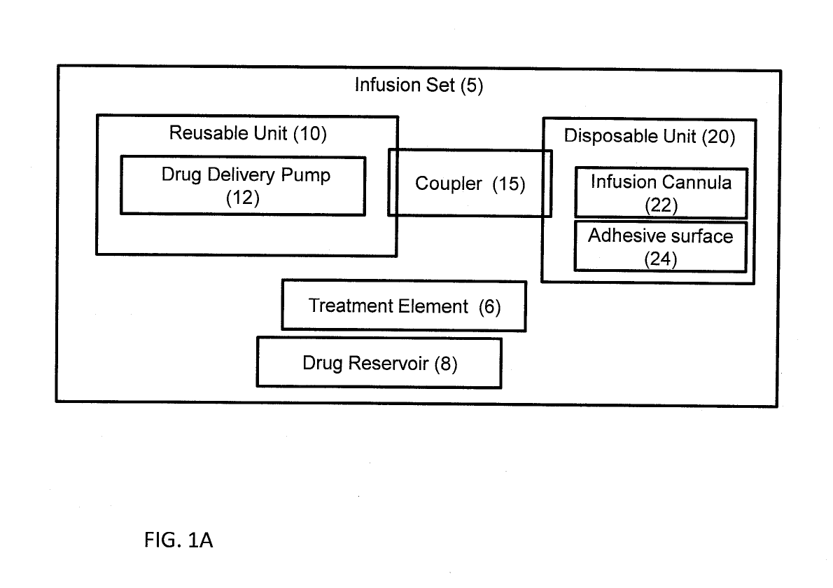

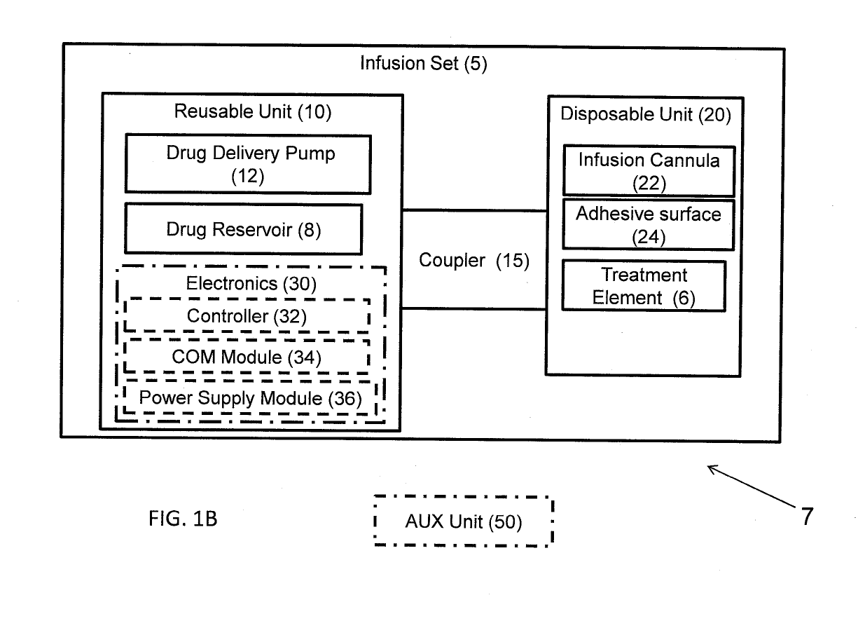

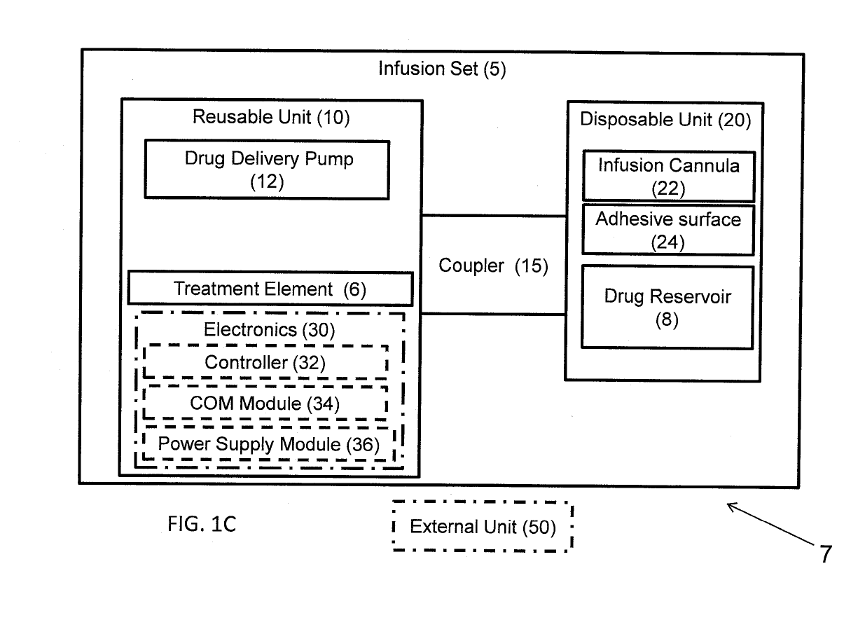

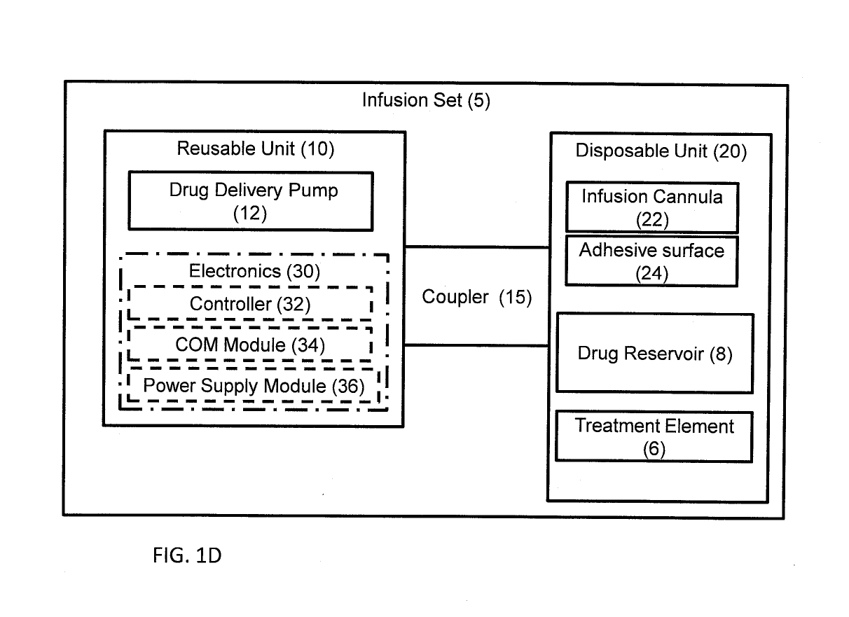

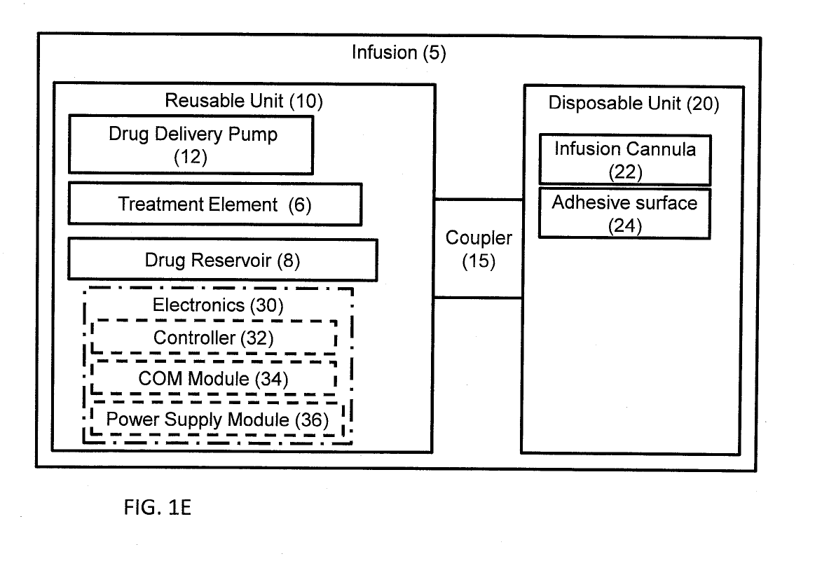

FIG. 1A shows a block diagram of an optional embodiment of the present invention showing infusion set and mechanical pump apparatus 5 comprising a reusable unit 10 and a disposable unit 20, a treatment element 6 and drug and/or medicament reservoir 8. Most preferably reusable unit and disposable unit may be coupled in a one to one relationship with coupler 15 so as to render the apparatus 5 operational and/or functional only when reusable unit 10 and disposable unit 20 are coupled in the correct manner. Optional embodiments of the present invention provide for the treatment element 6 and drug reservoir 8 to be disposed about and/or configured to be a part of and/or integrated with, either one or both of reusable unit 10 and disposable unit 20, or in any combination thereof. For example both treatment element 6 and reservoir 8 may be disposed within disposable unit 20, as shown in FIG. 1D. For example both treatment element 6 and reservoir 8 may be disposed within reusable unit 10, as shown in FIG. 1E. For example treatment element 6 may be disposed within disposable unit 20 while reservoir 8 disposed within reusable unit 10, as shown in FIG. 1B. For example treatment element 6 may be disposed within reusable unit 10 while reservoir 8 disposed within disposable unit 20, as shown in FIG. 1C.

Optionally drug reservoir 8 may be provided in a plurality of optional forms as a drug source for example including but not limited to vial, cartridge, IV bag, drug storage bag, or the like.

Most preferably reusable unit 10 comprises a drug delivery pump 12 that is most preferably a low cost manual pump that may for example take the form of a syringe like pump providing for pumping and/or delivering a drug and/or medicament from reservoir 8 to a drug delivery site (not shown). Optionally and most preferably pump 12 is provided in the form of a manual pump and/or mechanical pump. Optionally pump 12 may be provided in the form of a low cost electrical pump. Optionally pump 12 may be provided in the form of an automatic injection pen. Most preferably disposable unit 20 comprises a single use and/or single use period and/or disposable infusion catheter and/or infusion cannula 22 provided for delivering the drug from its reservoir 8 to the tissue site. Most preferably cannula 22 is coupled to a user for the duration of a single use period with an adhesive layer 24 covered with a laminate that may be to couple cannula 22 to the user's skin. Most preferably coupler 15 provides for coupling and aligning cannula 22 with corresponding opening in pump 12 so as to allow and ensure drug delivery flow from reservoir 8 to the drug delivery site (not shown). Optionally and preferably coupler 15 may further provide a drug delivery safety element provided to avoid accidental infusion. Optionally coupler 15 may further comprise an approval button and/or safety catch or the like member provided to ensure and/or approve drug delivery prior to infusion and/or manual pumping as a further safety and/or failsafe measure.

Optionally reusable unit 10 may further comprise an electronics module 30 provided for controlling and operating treatment element 6, for example provided in the form of a heating element. Electronics module 30 and/or circuitry may comprise a controller 32 for example a microprocessor, a power supply module 36 for example comprising rechargeable power source, and a communication module 34. Optionally communication module 34 may provide for wireless and/or wired and/or contactless communication with at least one or more optional auxiliary device 50 for example including but not limited to blood glucose monitor, computer, server, mobile communication device or the like, for example as shown in FIG. 1B

Optionally electronics module 30 may be provided to operate and control treatment element 6 when disposed on either of disposable unit 20 or reusable unit 10. Optionally module 30 may be coupled or otherwise functionally associated with treatment element 6.

Optionally treatment element 6 may be rendered operational with manual means for example including but not limited to user manual manipulation such as rubbing, pinching, twisting or the like manual or mechanical means for increasing local blood perfusion the drug infusion area.

FIG. 1B shows an optional infusion set system 7 according to an optional embodiment of the present invention comprising the drug delivery infusion set 5 and an optional auxiliary device 50. Most preferably auxiliary device 50 and infusion set 5 may be in contactless and/or wireless and/or wired communication via an optional communication module 34. Most preferably auxiliary device 50 comprises processing and display capabilities for interfacing with a user and/or device. Optionally, auxiliary device 50 may provide for controlling the amount of infused drug to be delivered.

FIG. 1C shows an optional configuration of infusion set 5 wherein treatment element 6 is disposed with the reusable unit 10. Optionally and preferably treatment element 6 may be powered with a electronics module 30.

FIG. 1D shows an optional configuration of infusion set 5 wherein both drug reservoir 8 and treatment element 6 are disposed within disposable unit 20. Most preferably coupler 15 provides for coupling and aligning pump 12 with reservoir 8 and cannula 22. Optionally coupler 15 may further provide for coupling electronic module 30 to treatment element 6.

FIG. 1E shows an optional configuration of infusion set 5 wherein both drug reservoir 8 and treatment element 6 are disposed within reusable unit 20. Most preferably coupler 15 provides for coupling and aligning pump 12 with reservoir 8 and cannula 22.

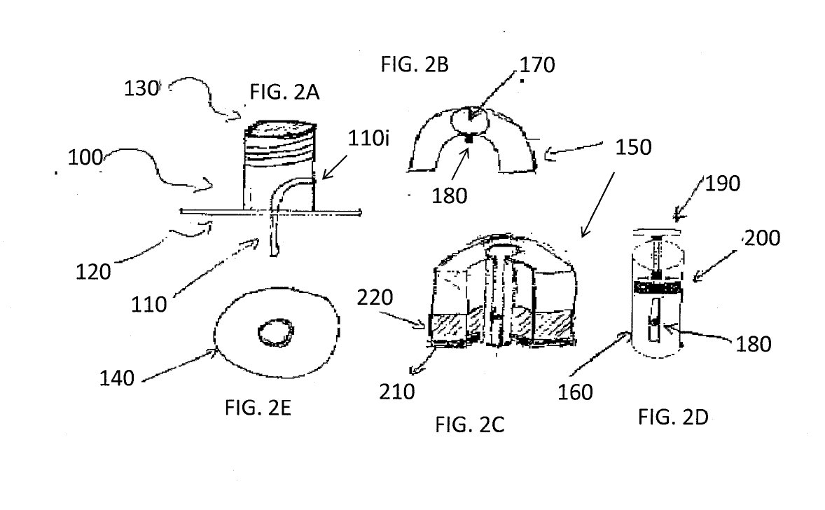

FIG. 2A-E show a schematic illustration of an optional embodiment of the present invention, similar to that described in FIG. 1A above, comprising an infusion set disposable member 100 comprising a catheter and/or cannula 110 for subcutaneous delivery of a medicament, that may be coupled to the body with an adhesive layer 120 provided for coupling to the injection site for a single use period, for example of up to about 3 days. Most preferably infusion set member 100 comprises a housing 130, provided for coupling a reusable pump member 200. Optionally and most preferably housing 130 comprises external threading, or the like coupling means, provided for coupling infusion set member 100 and pump member 200 in a one to one manner such that when coupled cannula 120 is aligned and rendered functional and can receive a flowing drug from pump member 200.

FIGS. 2B and 2C provide a top view and side view of functional unit 150 comprising treatment element 210 and drug reservoir 160. Most preferably functional unit 150 may be provided in a reusable, multiuse configuration or in a single use and/or disposable configuration. Functional unit 150 may be coupled to infusion set member 100 over corresponding external threading disposed about housing 130. Most preferably drug reservoir 160 and drug reservoir cavity 170 may be provide with an exit port 180 that is aligned with a corresponding entry port disposed about cannula 110i, in a one to one manner such that infusion set 100 is rendered functional only when exit port 180 and entry port 110i are successfully coupled and aligned. Optionally and preferably reservoir 160 may be placed in reservoir cavity 170. Optionally a third coupling member or drug safety element, for example in the form of a push button and/or safety catch and/or pin, that may be provided as a further failsafe prior to drug delivery and verification prior to drug infusion. For example coupler 15 (FIG. 1A-E) may be realized by way of coupling three members comprising, exit port 180, entry port 110i and a safety catch all of which must be aligned and functional to allow infusion.

Most preferably treatment element 210, for example provided in the form of a heating element, is provided such that it may provide treatment in the form of heat over the infusion site to modify pharmacodynamic and pharmacokinetic properties of the delivered drug. Preferably treatment element 210 may be controlled and rendered functional with optional electronic module 220 comprising power supply. Optionally electronic module 220 may comprise a controller, communication module and power supply module, for example as described in FIG. 1B-E.

FIG. 2D depicts a schematic illustration of a pump member 200 according to an optional embodiment of the present invention, for example as described in FIG. 1A. Manual pump member 200 comprises a plunger 190 that provides for manually pumping a flowing drug from drug reservoir 160 to cannula 110 at the infusion site. Optionally, plunger 190 may be a friction free plunger comprises a freely rotating ball to allow ease of manipulation of pump 200 with plunger 190 about housing 130 for example by the rotation of the circular disk 140 along the threads of the catheter housing, 130.

Optionally functional unit 150 may be integrated with infusion set housing 130, therein provided for single use. Optionally functional unit 150 may be integrated with pump housing 200, therein provided for multiuse.

Optionally plunger 190 or electronics module 220 may provide for setting the dosage to be dispensed and/or delivered through reservoir 160. Optionally dosage setting with plunger 190 may be provided by way of communication with an auxiliary device 50 (not shown).

Although particular embodiments have been disclosed herein in detail, this has been done by way of example and for purposes of illustration only, and is not intended to be limiting. In particular, it is contemplated by the inventors that various substitutions, alterations, and modifications may be made without departing from the spirit and scope of the disclosed embodiments. Other aspects, advantages, and modifications are considered to be within the scope of the disclosed and claimed embodiments, as well as other embodiments disclosed herein. The claims presented hereafter are merely representative of some of the embodiments disclosed herein. Other, presently unclaimed embodiments are also contemplated. The inventors reserve the right to pursue such embodiments in later claims and/or later applications claiming common priority.

There are many inventions described and illustrated herein. The present inventions are neither limited to any single aspect nor embodiment thereof, nor to any combinations and/or permutations of such aspects and/or embodiments. Moreover, each of the aspects of the present inventions, and/or embodiments thereof, may be employed alone or in combination with one or more of the other aspects of the present inventions and/or embodiments thereof. For the sake of brevity, many of those permutations and combinations will not be discussed separately herein.

Importantly, the Summary may not be reflective of or correlate to the inventions protected by the claims in this or continuation/divisional applications hereof Even where this Summary is reflective of or correlates to the inventions protected by the claims hereof, this Summary may not be exhaustive of the scope of the present inventions.

While the invention has been described with respect to a limited number of embodiment, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not described to limit the invention to the exact construction and operation shown and described and accordingly, all suitable modifications and equivalents may be resorted to, falling within the scope of the invention.

Having described a specific preferred embodiment of the invention with reference to the accompanying drawings, it will be appreciated that the present invention is not limited to that precise embodiment and that various changes and modifications can be effected therein by one of ordinary skill in the art without departing from the scope or spirit of the invention defined by the appended claims.

Further modifications of the invention will also occur to persons skilled in the art and all such are deemed to fall within the spirit and scope of the invention as defined by the appended claims.

While the invention has been described with respect to a limited number of embodiments, it will be appreciated that many variations, modifications and other applications of the invention may be made.

What is claimed is:

1. A device for delivering a therapeutic substance to the body of a mammal, comprising:
a treatment element for modifying the pharmacodynamic or pharmacokinetic properties of said therapeutic substance;
a reservoir for storing said therapeutic substance,
a manual infusion pump functionally associated with said reservoir for dispensing the contents of said reservoir; wherein said reservoir is in fluid communication and coupled with an infusion set catheter with a coupling member;
the infusion set comprising:
a catheter disposed in a catheter housing, the housing having an adhesive layer for coupling to a body, and wherein said catheter is in fluid communication and coupled with said reservoir about said coupling member.

2. The device according to claim 1, wherein said coupling member comprises a first member disposed about said catheter and a second member disposed about said reservoir.

3. The device of claim 2 wherein the device is rendered non-functional unless said first and second coupling member are properly coupled forming a fluid communication channel between said reservoir and said catheter.

4. The device according to claim 2, wherein said first and second coupling members are self-sealing.

5. The device according to claim 1, wherein said treatment element is disposed about said infusion set catheter housing or said a manual infusion pump.

6. The device according to claim 1, wherein said treatment element is provided in the form of a heating element.

7. The device according to claim 1, further comprising an electronic module for controlling said treatment element.

8. The device according to claim 7, wherein the controller is configured to activate the treatment element based on information regarding the therapeutic substance.

9. The device according to claim 7, wherein said electronic module comprises one or more fuses which is burned each time the treatment element is activated.

10. The device according to claim 7, wherein the controller executes a treatment protocol based on the information regarding the therapeutic substance.

11. The device according to claim 10, wherein the controller is configured to automatically activate the treatment profile element based on information of therapeutic substance volume delivered.

12. The device of claim 1 wherein said reservoir is provided in the form of a single use cartridge.

13. The device of claim 1 wherein said manual infusion pump provides for activating said treatment element.

14. The device of claim 1 wherein said manual infusion pump provides for determining the amount of drug dispensed from said reservoir.

15. The device according to claim 1, wherein the therapeutic substance is insulin.

16. The device according to claim 1, wherein the infusion set comprises identifying means that uniquely identifies the infusion set.

17. The device of claim 1 further comprising a drug safety element provided to avoid accidental infusion.

18. The device of claim 16 wherein said safety element comprises an approval button to approve the delivery of the selected drug to be infused.

19. The device of claim 3 wherein said coupling member further comprises a drug safety element to safeguarding against accidental drug infusion.

20. A system for delivering a therapeutic substance to the body of a mammal, comprising the device according to claim 1 in communication with an auxiliary device.

21. The system of claim 20 wherein said auxiliary device is selected from the group of mobile communication device, blood glucose monitor or computer.

22. The system of claim 20 wherein said auxiliary device determines the amount dispensed from said reservoir.
